SEQUENCE LISTING (5 sequences)

SEQ ID NO: 1 - DNA, 2898 bp, synthetic construct
  source 1..2898: mol_type = other DNA
gatgtggatgagtgtgtggaggggactgacaactgccacatcgatgctatctgccagaacaccccgaggtcatacaagtgcatctgcaagtctggctacacaggggacggcaaacactgcaaagacgtggatgagtgcgagcgagaggataatgcaggttgtgtgcatgactgtgtcaacatccctggcaattaccggtgtacctgctatgatggattccacctggcacatgacggacacaactgtctggatgtggacgagtgtgccgagggcaacggcggctgtcagcagagctgtgtcaacatgatgggcagctatgagtgccactgccgggaaggcttcttcctcagcgacaaccagcatacctgtatccagcggccagaagaaggaatgaattgcatgaacaagaaccacggctgtgcccacatttgccgggagacacccaaggggggtattgcctgtgaatgccgtcctggctttgagcttaccaagaaccaacgggactgtaaattgacatgcaactatggtaacggcggctgccagcacacgtgtgatgacacagagcagggtccccggtgcggctgccatatcaagtttgtgctccataccgacgggaagacatgcatcgagacctgtgctgtcaacaacgggggctgtgacagtaagtgccatgatgcagcgactggtgtccactgcacctgccctgtgggcttcatgctgcagccagacaggaagacgtgcaaagatatagatgagtgccgcttaaacaacgggggctgtgaccatatttgccgcaacacagtgggcagcttcgaatgcagttgcaagaaaggctataagcttctcatcaatgagaggaactgccaggatatagacgagtgttcctttgatcgaacctgtgaccacatatgtgtcaacacaccaggaagcttccagtgtctctgccatcgtggctacctgttgtatggtatcacccactgtggggatgtggatgaatgcagcatcaaccggggaggttgccgctttggctgcatcaacactcctggcagctaccagtgtacctgcccagcaggccagggtcggctgcactggaatggcaaagattgcacagagccactgaagtgtcagggcagtcctggggcctcgaaagccatgctcagctgcaaccggtctggcaagaaggacacctgtgccctgacctgtccctccagggcccgatttttgccagagtctgagaatggcttcacggtgagctgtgggacccccagccccagggctgctccagcccgagctggccacaatgggaacagcaccaactccaaccactgccatgaggctgcagtgctgtccattaaacaacgggcctccttcaagatcaaggatgccaaatgccgtttgcacctgcgaaacaaaggcaaaacagaggaggctggcagaatcacagggccaggtggtgccccctgctctgaatgccaggtcaccttcatccaccttaagtgtgactcctctcggaagggcaagggccgacgggcccggacccctccaggcaaagaggtcacaaggctcaccctggaactggaggcagaggtcagagccgaagaaaccacagccagctgtgggctgccctgcctccgacagcgaatggaacggcggctgaaaggatccctgaagatgctcagaaagtccatcaaccaggaccgcttcctgctgcgcctggcaggccttgattatgagctggcccacaagccgggcctggtagccggggagcgagcagagccgatggagtcctgtaggcccgggcagcaccgtgctgggaccaagtgtgtcagctgcccgcagggaacgtattaccacggccagacggagcagtgtgtgccatgcccagcgggcaccttccaggagagagaagggcagctctcctgcgacctttgccctgggagtgatgcccacgggcctcttggagccaccaacgtcaccacgtgtgcaggtcagtgcccacctggccaacactctgtagatgggttcaagccctgtcagccatgcccacgtggcacctaccaacctgaagcaggacggaccctatgcttcccttgtggtgggggcctcaccaccaagcatgaaggggccatttccttccaagactgtgacaccaaagtccagtgctccccagggcactactacaacaccagcatccaccgctgtattcgctgtgccatgggctcctatcagcccgacttccgtcagaacttctgcagccgctgtccaggaaacacaagcacagactttgatggctctaccagtgtggcccaatgcaagaatcgtcagtgtggtggggagctgggtgagttcactggctatattgagtcccccaactacccgggcaactacccagctggtgtggagtgcatctggaacatcaaccccccacccaagcgcaagatccttatcgtggtaccagagatcttcctgccatctgaggatgagtgtggggacgtcctcgtcatgagaaagaactcatccccatcctccattaccacttatgagacctgccagacctacgagcgtcccattgccttcactgcccgttccaggaagctctggatcaacttcaagacaagcgaggccaacagcgcccgtggcttccagattccctatgttacctatgatgaggactatgagcagctggtagaagacattgtgcgagatggccggctctatgcctctgaaaaccaccaggagattttaaaggacaagaagctcatcaaggccttctttgaggtgctagcccacccccagaactacttcaagtacacagagaaacacaaggagatgctgccaaaatccttcatcaagctgctccgctccaaagtttccagcttcctgaggccctacaaatag

SEQ ID NO: 2 - DNA, 29 bp, synthetic construct
  source 1..29: mol_type = other DNA
gggaatcacatcagtaactgagtgacttc

SEQ ID NO: 3 - DNA, 402 bp, synthetic construct
  source 1..402: mol_type = other DNA
atggcaaaattagagactgttactttaggtaacatcgggaaagatggaaaacaaactctggtcctcaatccgcgtggggtaaatcccactaacggcgttgcctcgctttcacaagcgggtgcagttcctgcgctggagaagcgtgttaccgtttcggtatctcagccttctcgcaatcgtaagaactacaaggtccaggttaagatccagaacccgaccgcttgcactgcaaacggttcttgtgacccatccgttactcgccaggcatatgctgacgtgaccttttcgttcacgcagtatagtaccgatgaggaacgagcttttgttcgtacagagcttgctgctctgctcgctagtcctctgctgatcgatgctattgatcagctgaacccagcgtattga

SEQ ID NO: 4 - DNA, 517 bp, synthetic construct
  source 1..517: mol_type = other DNA
ggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatg

SEQ ID NO: 5 - DNA, 3602 bp, synthetic construct
  source 1..3602: mol_type = other DNA
gggaatcacatcagtaactgagtgacttcggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggcctacccctacgacgtgcccgactacgccctggccaccacccatatgggatccgaattcatggctcattaccgtaatgacgatgtggatgagtgtgtggaggggactgacaactgccacatcgatgctatctgccagaacaccccgaggtcatacaagtgcatctgcaagtctggctacacaggggacggcaaacactgcaaagacgtggatgagtgcgagcgagaggataatgcaggttgtgtgcatgactgtgtcaacatccctggcaattaccggtgtacctgctatgatggattccacctggcacatgacggacacaactgtctggatgtggacgagtgtgccgagggcaacggcggctgtcagcagagctgtgtcaacatgatgggcagctatgagtgccactgccgggaaggcttcttcctcagcgacaaccagcatacctgtatccagcggccagaagaaggaatgaattgcatgaacaagaaccacggctgtgcccacatttgccgggagacacccaaggggggtattgcctgtgaatgccgtcctggctttgagcttaccaagaaccaacgggactgtaaattgacatgcaactatggtaacggcggctgccagcacacgtgtgatgacacagagcagggtccccggtgcggctgccatatcaagtttgtgctccataccgacgggaagacatgcatcgagacctgtgctgtcaacaacgggggctgtgacagtaagtgccatgatgcagcgactggtgtccactgcacctgccctgtgggcttcatgctgcagccagacaggaagacgtgcaaagatatagatgagtgccgcttaaacaacgggggctgtgaccatatttgccgcaacacagtgggcagcttcgaatgcagttgcaagaaaggctataagcttctcatcaatgagaggaactgccaggatatagacgagtgttcctttgatcgaacctgtgaccacatatgtgtcaacacaccaggaagcttccagtgtctctgccatcgtggctacctgttgtatggtatcacccactgtggggatgtggatgaatgcagcatcaaccggggaggttgccgctttggctgcatcaacactcctggcagctaccagtgtacctgcccagcaggccagggtcggctgcactggaatggcaaagattgcacagagccactgaagtgtcagggcagtcctggggcctcgaaagccatgctcagctgcaaccggtctggcaagaaggacacctgtgccctgacctgtccctccagggcccgatttttgccagagtctgagaatggcttcacggtgagctgtgggacccccagccccagggctgctccagcccgagctggccacaatgggaacagcaccaactccaaccactgccatgaggctgcagtgctgtccattaaacaacgggcctccttcaagatcaaggatgccaaatgccgtttgcacctgcgaaacaaaggcaaaacagaggaggctggcagaatcacagggccaggtggtgccccctgctctgaatgccaggtcaccttcatccaccttaagtgtgactcctctcggaagggcaagggccgacgggcccggacccctccaggcaaagaggtcacaaggctcaccctggaactggaggcagaggtcagagccgaagaaaccacagccagctgtgggctgccctgcctccgacagcgaatggaacggcggctgaaaggatccctgaagatgctcagaaagtccatcaaccaggaccgcttcctgctgcgcctggcaggccttgattatgagctggcccacaagccgggcctggtagccggggagcgagcagagccgatggagtcctgtaggcccgggcagcaccgtgctgggaccaagtgtgtcagctgcccgcagggaacgtattaccacggccagacggagcagtgtgtgccatgcccagcgggcaccttccaggagagagaagggcagctctcctgcgacctttgccctgggagtgatgcccacgggcctcttggagccaccaacgtcaccacgtgtgcaggtcagtgcccacctggccaacactctgtagatgggttcaagccctgtcagccatgcccacgtggcacctaccaacctgaagcaggacggaccctatgcttcccttgtggtgggggcctcaccaccaagcatgaaggggccatttccttccaagactgtgacaccaaagtccagtgctccccagggcactactacaacaccagcatccaccgctgtattcgctgtgccatgggctcctatcagcccgacttccgtcagaacttctgcagccgctgtccaggaaacacaagcacagactttgatggctctaccagtgtggcccaatgcaagaatcgtcagtgtggtggggagctgggtgagttcactggctatattgagtcccccaactacccgggcaactacccagctggtgtggagtgcatctggaacatcaaccccccacccaagcgcaagatccttatcgtggtaccagagatcttcctgccatctgaggatgagtgtggggacgtcctcgtcatgagaaagaactcatccccatcctccattaccacttatgagacctgccagacctacgagcgtcccattgccttcactgcccgttccaggaagctctggatcaacttcaagacaagcgaggccaacagcgcccgtggcttccagattccctatgttacctatgatgaggactatgagcagctggtagaagacattgtgcgagatggccggctctatgcctctgaaaaccaccaggagattttaaaggacaagaagctcatcaaggccttctttgaggtgctagcccacccccagaactacttcaagtacacagagaaacacaaggagatgctgccaaaatccttcatcaagctgctccgctccaaagtttccagcttcctgaggccctacaaatagctcgagtgagatctgactgaaaaaaaaaaaaaaaaaaaaaaaaaaaaaagtttaaacactagtccgctgagcaataa

(19) United States
(12) Patent Application Publication
(10) Pub. No.: US 2024/0398721 A1
(43) Pub. Date: Dec. 5, 2024

(54) COMPOSITIONS AND METHODS FOR STIMULATING HAIR GROWTH USING NANOCAPSULE-BASED DRUG DELIVERY SYSTEM

(71) Applicant: VL Biotech Inc., New Taipei (TW)

(72) Inventors: Hsin-Yi LIN, New Taipei (TW); Stanley KUNG, New Taipei (TW)

(21) Appl. No.: 18/328,534

(22) Filed: Jun. 2, 2023

(51) Int. Cl. A61K 9/51; A61P 17/14; C07K 14/005; C07K 14/485; C12N 15/85

(52) U.S. Cl. CPC A61K 9/5184; A61P 17/14; C07K 14/005; C07K 14/485; C12N 15/85; A61K 48/00; C12N 2840/203

(57) ABSTRACT

Disclosed herein are compositions and methods with enhanced capability to stimulate hair growth. The compositions are loaded in a nanocarrier for passive skin delivery. The composition comprises a first vector and a second vector. The first vector encodes one or more mRNAs. The one or more mRNAs encode a peptide with SCUBE3 activity and are labeled with one or more capsid protein tags. The second vector encodes one or more capsid proteins. The one or more capsid proteins bind to the one or more capsid protein tags on the one or more mRNA.

### REFERENCE TO AN ELECTRONIC SEQUENCE LISTING

The content of the electronic sequence listing (23HPUS0013_sequencing list.xml; Size: 12 KB; and Date of Creation: May 29, 2023) is herein incorporated by reference in its entirety.

### FIELD OF THE INVENTION

The present invention relates to compositions and methods of use, in particular to therapeutic compositions with enhanced capability to stimulate hair growth in a human or animal subject, which are delivered by using nanocapsule-based drug delivery system. Compositions and methods of the present invention are particularly applicable to a range of disorders, including androgenetic alopecia, telogen effluvium, and alopecia areata and related conditions.

### BACKGROUND OF THE INVENTION

Alopecia, also known as hair loss, is occurred due to different causes, such as age, genetics, hormonal imbalance, medication, and cancer treatment. The current treatments for hair loss include taking medicine like finasteride and minoxidil, wearing a wig, transplanting hair, etc.

Recently, researchers discovered that SCUBE3, a signaling mesenchymal niche factor, can stimulate hair growth. Regardless of the hair growth outcome of using animal study looks promising, it requires daily intradermal injections to deliver SCUBE3, which will limit the therapeutic effect since SCUBE3 may poorly target the dermal papilla cells residing at the deepest of a hair follicle.

### SUMMARY OF THE INVENTION

The present disclosure provides a composition for stimulating hair growth. The composition comprises a first vector and a second vector. The first vector comprises the nucleotide sequence as denoted by SEQ ID NO: 1 and a capsid protein recognition sequence. The first vector encodes one or more mRNAs encoding a peptide with SCUBE3 activity and the one or more mRNAs bind to one or more capsid protein tags. The second vector comprises one or more nucleotide sequences encoding one or more capsid proteins. The one or more capsid proteins are capable of self-assembling into one or more virus-like particles that encapsulate the one or more mRNAs such that the composition is fabricated as nanocapsule.

The present disclosure also provides a composition for stimulating hair growth. The composition comprises a first vector and a second vector. The first vector comprises the nucleotide sequence as denoted by SEQ ID NO: 5. The second vector comprises the nucleotide sequence as denoted by SEQ ID NO: 3. The nucleotide sequence of SEQ ID NO: 3 encodes one or more capsid proteins, and the one or more capsid proteins are capable of self-assembling into one or more virus-like particles that encapsulate one or more mRNAs encoded by the nucleotide sequence of SEQ ID NO: 5 such that the composition is fabricated as nanocapsule.

The present disclosure further provides a method for stimulating hair growth in a subject. The method includes administering a composition comprising one or more nanocapsules to the subject. The one or more nanocapsules are generated by a first vector and a second vector. The first vector encodes one or more mRNAs, and the one or more mRNAs encode a peptide with SCUBE3 activity. The one or more mRNAs are labeled with one or more capsid protein tags. The second vector comprises one or more nucleotide sequences encoding one or more capsid proteins. The one or more capsid proteins are capable of self-assembling into one or more virus-like particles that encapsulate the one or more mRNAs such that the composition is fabricated as the one or more nanocapsules.

### BRIEF DESCRIPTION OF THE DRAWINGS

FIG. 1 illustrates an electrophoresis result showing the recognition of SCUBE3 production via a non-SCUBE3-capable cell line.

FIG. 2 illustrates the results of the hair growth stimulation test, (A) is a photo taken before the administration of the Composition 1, (B) is a photo taken after the administration of the Composition 1 for seven days.

### DETAILED DESCRIPTION OF THE PREFERRED EMBODIMENTS

As used herein, the term “vector” used in reference to delivery of components refers to a nucleic acid capable of transporting between different genetic environments another nucleic acid to which it has been operatively linked. The term “express” in the context of the disclosure refers to transcription and/or translation of a specific nucleotide sequence driven by its promoter.

The disclosure relates to one or more nucleotide sequences encoding a peptide with SCUBE3 activity, which particularly can be assembled into nanocapsule in bacteria, for stimulating hair growth. Thus, the one or more nucleotide sequences can be incorporated into pharmaceutical and/or therapeutic compositions suitable for administration.

The present disclosure discloses a first vector and a second vector, the first vector encodes one or more messenger ribonucleic acids (messenger RNAs, mRNAs) encoding a peptide with SCUBE3 activity. The one or more mRNAs are further labeled with one or more capsid protein tags. The second vector encodes one or more capsid proteins (CPs), which may be self-assembled to form one or more hollow virus-like particles (virus-like particles, VLPs), then bind to and provide domain specificity to the one or more capsid protein tags on the one or more mRNAs, such that the one or more mRNAs are capable of being encapsulated in the one or more VLPs. Thus, the one or more mRNAs encoding the peptide with SCUBE3 activity are carried in nanocarrier and will be delivered via nanocapsules after administration.

In some aspects, the pharmaceutical and/or therapeutic compositions comprise bio-products or compounds transcribed or translated from the first vector and the second vector described in the present disclosure.

The pharmaceutical and/or therapeutic composition may be used to stimulate hair growth. Therefore, in one aspect, it is contemplated that the compositions and methods of the disclosure are used to treat and/or prevent disorders of non-scarring hair loss, such as androgenetic alopecia, telogen effluvium, and alopecia areata.

In one aspect, the disclosure relates to one or more nanocapsule containing bacteria, which is assembled from one or more bio-products transcribed or translated from the first vector and the second vector. The bio-products may be either the mRNAs or the capsid proteins.

In another aspect, the disclosure relates to one or more nanocapsule assembled from one or more bio-products transcribed or translated from the first vector and the second vector. The bio-products may be either the mRNAs or the capsid proteins.

In yet another aspect, the disclosure relates to one or more VLPs assembled from one or more bio-products transcribed or translated from the first vector and the second vector. The bio-products may be either the mRNAs or the capsid proteins.

In further aspect, the disclosure provides a use of the pharmaceutical and/or therapeutic composition of the disclosure in a preparation of a medicament for a treatment of disorders of non-scarring hair loss, such as androgenetic alopecia, telogen effluvium, and alopecia areata.

In another aspect, the disclosure provides a method for treating and/or preventing disorders of non-scarring hair loss, such as androgenetic alopecia, telogen effluvium, or alopecia areata, which comprises administering the pharmaceutical and/or therapeutic composition of the disclosure or the preparation thereof.

In one embodiment, the first vector comprises the nucleotide sequence as denoted by SEQ ID NO: 1, and a capsid protein recognition sequence. The first vector is transcribed to form mRNA with the one or more capsid protein tags, which encodes a peptide with SCUBE3 activity and can be used as a translation template in a cell of a human or animal subject to produce a peptide with SCUBE3 activity in the human or animal subject's body, to endogenously produce SCUBE3 in vivo. In addition, the one or more capsid protein tags can bind to a specific region of one or more capsid proteins because of a secondary structure formed by the capsid protein recognition sequence. In one embodiment, the capsid protein recognition sequence comprises the nucleotide sequence as denoted by SEQ ID NO: 2, and the one or more capsid protein tags formed by the capsid protein recognition sequence has a hairpin structure.

Further, the second vector comprises one or more nucleotide sequences encoding the one or more capsid proteins. In one embodiment, the one or more capsid proteins are shell proteins derived from bacteriophages, such as AP205 phage, Qbeta phage, MS2 phage, P22 phage and the like. In another embodiment, the second vector comprises the nucleotide sequence as denoted by SEQ ID NO: 3, and the one or more capsid proteins encoded by the nucleotide sequence of SEQ ID NO: 3 can be bound with the one or more capsid protein tags formed by the nucleotide sequence of SEQ ID NO: 2.

The first vector of the composition expresses the one or more mRNAs encoding the peptide with SCUBE3 activity and the second vector of the composition expresses the one or more capsid proteins. The one or more capsid protein tags on the one or more mRNAs facilitate the combination of the capsid proteins and the one or more mRNAs. Consequently, the one or more capsid proteins are self-assembled into the one or more virus-like particles which can be utilized to deliver the one or more RNAs encoding the peptide with SCUBE3 activity in the form of nanocapsules.

The first vector may further comprise an internal ribosome entry site (IRES) containing the nucleotide sequence as denoted by SEQ ID NO: 4. The internal ribosome entry site is placed between the nucleotide sequence of SEQ ID NO: 1 and the capsid protein recognition sequence. The first vector may include a promoter and a terminator for RNA polymerase to perform transcription.

In another embodiment, the composition of the disclosure comprises a first vector and a second vector. The first vector comprises the nucleotide sequence as denoted by SEQ ID NO: 5 encoding one or more capsid protein tags and one or more mRNAs which encodes a peptide with SCUBE3 activity. The second vector comprises the nucleotide sequence as denoted by SEQ ID NO: 3 encoding one or more capsid proteins. The one or more capsid proteins encoded by the nucleotide sequence of SEQ ID NO: 3 can be combined with the one or more capsid protein tags formed by the nucleotide sequence of SEQ ID NO: 5.

The disclosure further provides a nanocapsule-based drug delivery system for delivering mRNA into the body of the human or animal subject to stimulate hair growth of the human or animal subject. In one embodiment, the nanocapsule-based drug delivery system is achieved by various administrations, which are used to enable delivery of the compositions to the desired site of biological action. These administrations include, but are not limited to oral routes, intraduodenal routes, parenteral injection (including intravenous, subcutaneous, intraperitoneal, intramuscular, intravascular or infusion), topical and rectal administration.

In one embodiment, the first vector and the second vector are transformed into a transformed cell, and the genetic information of the first vector and the second vector are expressed through the physiological mechanism of the transformed cell. In an example, the transformed cells are bacterial cells. In the process of the transformation, the first vector and the second vector are either transcribed into mRNAs or translated into capsid proteins; the bio-products are then self-assembled into the VLPs, encapsulated in the bacterial cells. In an alternative example, the transformed cells are probiotics. In the process of the transformation, the first vector and the second vector are either transcribed into mRNAs or translated into capsid proteins; the bio-products are then self-assembled into the VLPs, encapsulated in the probiotics.

Specifically, the first vector is expressed by the transformed cell to produce the one or more mRNAs with the one or more capsid protein tags, and the one or more mRNAs encode a peptide with SCUBE3 activity in the body of the human or animal subject. The second vector is expressed by the transformed cell to form one or more capsid proteins. The products of the first vector and the second vector can be self-assembled into nanocapsule in the transformed cell by binding of the one or more capsid protein tags to the one or more capsid proteins.

The composition of the disclosure can promote the body of the human or animal subject to produce the peptides with SCUBE3 activity, which are more similar to endogenous SCUBE3 than artificially synthesized SCUBE3 derivatives. The SCUBE3 produced in vivo is effectively capable of activating the neighboring follicle niche fibroblast, so as to enable therapeutic effects by stimulating hair growth.

In one embodiment, the transformed cells are bacterial cells, which can be easily obtained and cultured from bacterial cultures. The bacterial cells may be Escherichia coli.

The following examples are disclosed to demonstrate that the composition of the disclosure is able to endogenously produce SCUBE3 in vivo, causing an increase of hair growth. The following examples are only for the purpose of illustration, and the scope of the disclosure is not limited by the examples. Those skilled in the art can utilize the disclosure and teachings of the disclosure to create other embodiments, aspects and variations without undue experimentation.

### Example 1

The Composition 1 is prepared by transforming the first vector and the second vector into Escherichia coli cells. The first vector adopts pT7CFE1-NHA vector and contains the nucleotide sequence of SEQ ID NO: 5. The second vector adopts pCDF-1b vector and contains the nucleotide sequence of SEQ ID NO: 3. Then the Composition 1 is administered to eukaryotic host cells, HEK293 cells, by adding into the culture medium of HEK293 cells and cultured for 12 hours.

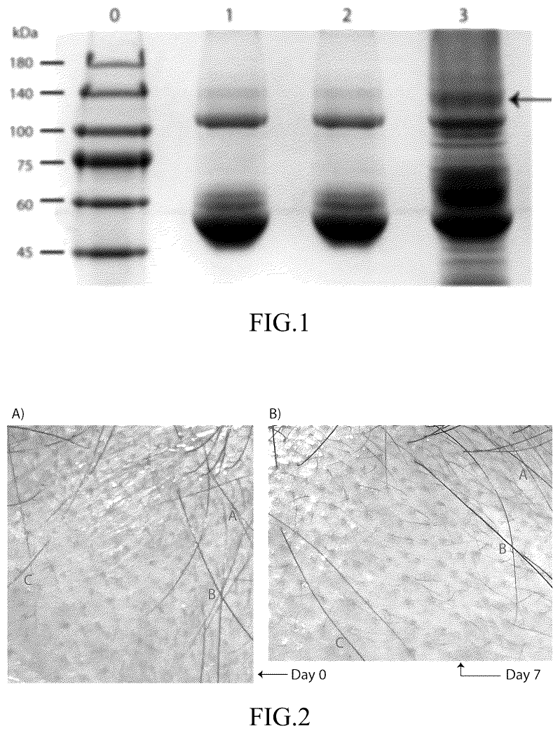

The overnight cultured medium was then collected for visualization on electrophoresis analysis. In FIG. 1, the first channel is filled with the protein ladder, the second channel is filled with the cultured medium of HEK293 cells without administering the Composition 1 as a negative control group, the third channel is filled with the cultured medium of HEK293 cells administered the nano-capsule without containing the nucleotide sequence of SEQ ID NO: 5 as a positive control group, and the fourth channel is filled with the culture medium of HEK293 cells administered the Composition 1. As shown in FIG. 1, a clear ˜130 kDa band shown alone in the fourth channel, which indicates that the HEK293 cells inherently without SCUBE3 activity will produce SCUBE3 after being cultured with the Composition 1 for 12 hours. The result demonstrates that the Composition 1 can promote eukaryotic cells to produce SCUBE3.

### Example 2

Composition 1 was tested on the human scalp to demonstrate the effect of growing hair. The volunteer externally applied the skin lotion which contains the composition 1 on a test zone of the scalp twice daily for seven days. FIG. 2. are the photos of the test zone of the scalp under magnified view taken before the use of skin lotion (left) and after the use of skin lotion for seven days (right).

It can be clearly seen from FIG. 2 that after seven days of using skin lotion, newly-growth tiny hairs bumped out of the follicle, indicating that the Composition 1 can be passive skin delivery to the 10 skin cells and stimulating hair growth, hence being expected to be useful for treating disorders of non-scarring hair loss.

What is claimed is:

1. A composition for stimulating hair growth, comprising:
a first vector, comprising a nucleotide sequence as denoted by SEQ ID NO: 1 and a capsid protein recognition sequence, wherein the first vector encodes one or more mRNAs encoding a peptide with SCUBE3 activity and the one or more mRNAs are labeled with one or more capsid protein tags; and
a second vector, comprising one or more nucleotide sequences encoding one or more capsid proteins, wherein the one or more capsid proteins are capable of self-assembling into one or more virus-like particles that encapsulate the one or more mRNAs such that the composition is fabricated as nanocapsule.

2. The composition according to claim 1, wherein the capsid protein recognition sequence in the first vector comprises a nucleotide sequence as denoted by SEQ ID NO: 2, and the second vector comprises the one or more nucleotide sequences as denoted by SEQ ID NO: 3.

3. The composition according to claim 1, wherein the first vector further comprises an internal ribosome entry site (IRES) between the nucleotide sequence of SEQ ID NO: 1 and the capsid protein recognition sequence.

4. A composition for stimulating hair growth, comprising:
a first vector, comprising a nucleotide sequence as denoted by SEQ ID NO: 5; and
a second vector, comprising a nucleotide sequence as denoted by SEQ ID NO: 3;
wherein the nucleotide sequence of SEQ ID NO: 3 encodes one or more capsid proteins, and the one or more capsid proteins are capable of self-assembling into one or more virus-like particles that encapsulate one or more mRNAs encoded by the nucleotide sequence of SEQ ID NO: 5 such that the composition is fabricated as nanocapsule.

5. A method for stimulating hair growth in a subject, comprising:
administering a composition comprising one or more nanocapsules to the subject, wherein the one or more nanocapsules are formed from a first vector and a second vector, the first vector encoding one or more mRNAs, wherein the one or more mRNAs encode a peptide with SCUBE3 activity, and the one or more mRNAs are labeled with one or more capsid protein tags, the second vector comprising one or more nucleotide sequences encoding one or more capsid proteins, wherein the one or more capsid proteins are capable of self-assembling into one or more virus-like particles that encapsulate the one or more mRNAs such that the composition is fabricated as the one or more nanocapsules.

6. The method according to claim 5, wherein the first vector comprises a nucleotide sequence as denoted by SEQ ID NO: 1 and a capsid protein recognition sequence.

7. The method according to claim 6, wherein the capsid protein recognition sequence in the first vector comprises a nucleotide sequence as denoted by SEQ ID NO: 2, and the second vector comprises the one or more nucleotide sequences as denoted by SEQ ID NO: 3.

8. The method according to claim 6, wherein the first vector further comprises an internal ribosome entry site (IRES) between the nucleotide sequence of SEQ ID NO: 1 and the capsid protein recognition sequence.

9. The method according to claim 5, wherein the first vector comprises a nucleotide sequence as denoted by SEQ ID NO: 5, and the second vector comprises the one or more nucleotide sequences as denoted by SEQ ID NO: 3.
